(19) United States
(12) Patent Application Publication
(10) Pub. No.: US 2024/0115627 A1
(43) Pub. Date: Apr. 11, 2024

(54) USE OF CLOSTRIDIUM GHONII SPORE COMBINED WITH PEMBROLIZUMAB

(71) Applicant: Shandong Xinchuang Biotechnology Co., Ltd., Shandong (CN)

(72) Inventors: Yong Wang, Shandong (CN); Hong Zhu, Shandong (CN); Wenhua Zhang, Shandong (CN); Yanqiu Xing, Shandong (CN); Dan Wang, Shandong (CN); Yuanyuan Liu, Shandong (CN); Shaopeng Wang, Shandong (CN); Jiahui Zheng, Shandong (CN); Rong Zhang, Shandong (CN); Xiaonan Li, Shandong (CN); Xinglu Xu, Shandong (CN); Shengbiao Jiang, Shandong (CN); Lichao Xing, Shandong (CN); Yuxia Gao, Shandong (CN); Shili Shao, Shandong (CN); Ting Han, Shandong (CN)

(21) Appl. No.: 18/546,077

(22) PCT Filed: Oct. 9, 2022
(86) PCT No.: PCT/CN2022/124020

§ 371 Date: Aug. 10, 2023

(30) Foreign Application Priority Data
Oct. 9, 2021 (CN) 202111177854.3

(51) Int. Cl. A61K 35/742; A61K 9/00; A61K 9/19; A61P 35/00; C07K 16/28

(52) U.S. Cl. CPC A61K 35/742; A61K 9/0019; A61K 9/19; A61P 35/00; C07K 16/2827; A61K 2039/505

(57) ABSTRACT

The present disclosure relates to use of a Clostridium ghonii spore combined with pembrolizumab in cancer treatment. It is found for the first time that the Clostridium ghonii spore combined with pembrolizumab can significantly improve a curative effect of colon cancer and reduce a dose of the pembrolizumab, and thus is efficient and low-toxic. Oncolysis by Clostridium ghonii can affect immunogenicity of a tumor microenvironment (TME) by various ways, converts an immunosuppressive state of the TME into an immune-activated state, adjusts the immunosuppressive TME, and breaks an immune tolerance. An optimal combination of the Clostridium ghonii spore and the pembrolizumab thoroughly removes about 20% of mouse tumor tissues. A benefit range of patients with tumors treated by a PD-1 antibody is expanded. The combination even has an obvious curative effect on patients failed the treatment by the PD-1 antibody.

### CROSS REFERENCE TO RELATED APPLICATION

The present application is a national stage application of International Patent Application No. PCT/CN2022/124020, filed on Oct. 9, 2022, which claims priority to the Chinese Patent Application No. CN202111177854.3, filed with the China National Intellectual Property Administration (CNIPA) on Oct. 9, 2021, and entitled “USE OF CLOSTRIDIUM GHONII SPORE COMBINED WITH PD-1 ANTIBODY”, which is incorporated herein by reference in its entirety.

### TECHNICAL FIELD

The present disclosure belongs to the technical field of biomedicine, and in particular relates to use of Clostridium ghonii combined with pembrolizumab.

### BACKGROUND

Tumor microenvironment (TME) is a local steady-state environment composed of tumor cells, stromal cells (including fibroblasts, immune and inflammatory cells, and some vascular endothelial cells, etc.), extracellular matrix and biomolecules infiltrated therein during a tumor growth process. The TME provides necessary material basis for tumorigenesis, development, invasion, etc., and regulates and controls biological behaviors such as tumor metastasis and relapse. Meanwhile, the TME can increase drug resistance and radiation resistance of tumors and reduces a treatment effect. Immunomodulation in the TME has an important function in tumorigenesis and development and leads to local immunosuppression in tumors through a variety of mechanisms. Therefore, how to regulate a TME-based immunotherapy strategy and remodel an active immune microenvironment are important for an anti-tumor therapy.

Pembrolizumab is a PD-1 inhibitor. PD-1 is an important immunosuppressive molecule, thus a PD-1 antibody shows a significant anti-tumor effect clinically, including a lasting therapeutic effect on a patient in a late stage metastasis. It is generally accepted that sensitivity to immune checkpoint blockers depends on tumor neoantigen burden and an infiltration degree and a composition of immune cells in the TME. Unfortunately, most common cancers do not exhibit extensive mutation and immune cell infiltration. Thus, tumors are insensitive to the immune checkpoint inhibitors with a low response rate and the immune checkpoint inhibitors are effective to only 20% of patients. Therefore, it is also one of major research directions at present to develop methods enabling the tumors to be more sensitive to immunotherapy.

Clostridium ghonii is a strict anaerobe, has a tumor-oriented hypoxic property, and can only colonize in a tumor hypoxia area. Oncolysis by the Clostridium ghonii can recruit a large number of immune cells to infiltrate into the TME, including innate immune cells of dendritic cells, neutrophils, and macrophages, specific immune cells of CD³⁺ T, CD⁴⁺ T, and CD⁵⁺ T cells, and thus changes the infiltration degree and the composition of the immune cells in the TME. At the same time, the oncolysis by the Clostridium ghonii enhances expressions of cytokines and chemokines such as TNF-α, IFN-γ, and IL-6 in tumors. It can be seen that oncolysis by Clostridium ghonii can affect immunogenicity of a tumor microenvironment (TME) by various ways, converts an immunosuppressive state of the TME into an immune-activated state, adjusts the immunosuppressive TME, and breaks an immune tolerance. Therefore, it is possible to effectively prevent tumors by the Clostridium ghonii combined with the immune checkpoint inhibitor.

### SUMMARY

According to deficiencies of the prior art, the present disclosure provides use of a Clostridium ghonii spore combined with a PD-1 antibody.

The present disclosure adopts the following technical solutions.

Use of a Clostridium ghonii spore combined with pembrolizumab in preparing a pharmaceutical product for treating colon cancer is provided.

A drug for treating colon cancer is provided, where the drug includes active ingredients of a Clostridium ghonii spore and pembrolizumab.

According to the present disclosure, preferably, the Clostridium ghonii may be a Clostridium ghonii MW-DCG-LCv-26 strain or a strain obtained after domestication of the Clostridium ghonii; and the Clostridium ghonii MW-DCG-LCv-26 is deposited in the National Measurement Institute, Australia, with a date of deposit of Apr. 18, 2012 and a deposit number of V12/001486. Other preferred strains may be an MW-DCG-HNCv-18 strain, deposited in the National Measurement Institute, Australia, with a date of deposit of Apr. 18, 2012 and a deposit number of V12/001485; or an MW-DCG-CCv-17 strain, deposited in the National Measurement Institute, Australia, with a date of deposit of Apr. 18, 2012 and a deposit number of V12/001487.

According to the present disclosure, preferably, the Clostridium ghonii may be in a spore form.

Preferably, the spore form of the Clostridium ghonii may be freeze-dried powder with an auxiliary material of 1% sucrose.

Preferably, the pembrolizumab is a PD-1 antibody injection.

More preferably, a freeze-drying procedure of the Clostridium ghonii spore freeze-dried powder for injection includes: freezing at −40° C. for 4 h, vacuumizing at −35° C. for 10 min, and freeze-drying at −30° C. for 10 min, −25° C. for 10 min, −20° C. for 26 h, −15° C. for 2 h, −10° C. for 10 min, −5° C. for 10 min, 0° C. for 10 min, 10° C. for 2 h, 15° C. for 10 min, 20° C. for 3 h and 27° C. for 3 h.

A preferred pharmaceutical combination of the present disclosure is 1×10⁷ CFU of the Clostridium ghonii spore freeze-dried powder combined with 0.2 mg of a pembrolizumab injection at a concentration of 1 mg/mL.

Further preferably, the pembrolizumab injection has a solvent of a sodium chloride injection with a mass percentage of 0.9%; and the Clostridium ghonii spore freeze-dried powder has a solvent of sterile water for injection and a sodium chloride injection with a mass percentage of 0.9%.

Further preferably, the Clostridium ghonii spore combined with pembrolizumab is used in a sequence of the Clostridium ghonii spore and then the pembrolizumab.

The present disclosure has the following beneficial effects:

It is found for the first time that the Clostridium ghonii spore combined with pembrolizumab can significantly improve a curative effect of colon cancer and reduce a dose of the pembrolizumab, and thus is efficient and low-toxic. Oncolysis by Clostridium ghonii can affect immunogenicity of a tumor microenvironment (TME) by various ways, converts an immunosuppressive state of the TME into an immune-activated state, adjusts the immunosuppressive TME, and breaks an immune tolerance. An optimal combination of the Clostridium ghonii spore and the pembrolizumab thoroughly removes about 20% of mouse tumor tissues. A benefit range of patients with tumors treated by a PD-1 antibody is expanded. The combination even has an obvious curative effect on patients failed the treatment by the PD-1 antibody.

### BRIEF DESCRIPTION OF THE DRAWINGS

FIG. 1A and FIG. 1B respectively show tumor weight and a tumor inhibition rate based on tumor weight of each group in a CT26.WT colon cancer tumor-bearing mouse model treated with a Clostridium ghonii spore in Example 1; and data are expressed as: * indicates P<0.05 compared with a Control group; and ** indicates P<0.01 compared with the Control group;

FIG. 2A and FIG. 2B respectively show tumor weight and a tumor inhibition rate based on tumor weight of each group in a MC38 colon cancer tumor-bearing mouse model treated with a Clostridium ghonii spore combined with pembrolizumab in Example 2; and data are expressed as: * indicates P<0.05; and ** indicates P<0.01;

FIG. 3 shows a tumor volume change of each group in a MC38 colon cancer tumor-bearing mouse model treated with a Clostridium ghonii spore combined with pembrolizumab in Example 2; and data are expressed as: * indicates P<0.05 compared with a Control group; ** indicates P<0.01 compared with the Control group; ^{#} indicates P<0.05 in a C. ghonii+Pembrolizumab group compared with a C. ghonii group; and ^{##} indicates P<0.01 in the C. ghonii+Pembrolizumab group compared with the C. ghonii group;

FIG. 4 shows a tumor inhibition rate based on tumor volume at an experimental endpoint in a MC38 colon cancer tumor-bearing mouse model treated with a Clostridium ghonii spore combined with pembrolizumab in Example 2; and data are expressed as: * indicates P<0.05; and ** indicates P<0.01;

FIGS. 5A-5C show a tumor inhibition rate based on tumor volume of each batch in a MC38 colon cancer tumor-bearing mouse model treated with a Clostridium ghonii spore combined with pembrolizumab in Example 2; specifically, FIG. 5A shows experiment batch 1, FIG. 5B shows experiment batch 2 and FIG. 5C shows experiment batch 3;

FIG. 6 shows morphological photos of tumors of each group in a MC38 colon cancer tumor-bearing mouse model treated with a Clostridium ghonii spore combined with pembrolizumab in Example 2;

FIG. 7 shows tumor weight of each group in a MC38 colon cancer tumor-bearing mouse model treated with a Clostridium ghonii spore combined with pembrolizumab in a different administration sequence in Example 3; and

FIG. 8 shows a tumor volume change of each group in a MC38 colon cancer tumor-bearing mouse model treated with a Clostridium ghonii spore combined with pembrolizumab in a different administration sequence in Example 3.

### DETAILED DESCRIPTION OF THE EMBODIMENTS

The present disclosure will be further described in detail below in conjunction with specific examples. The described examples are only a part of technical solutions listed in the present disclosure for the convenience of the public to understand. These examples are only used to illustrate the present disclosure and not to limit the protection scope of the present disclosure. The protection scope of the present disclosure is defined by the claims.

### Example 1 Therapeutic Effect of Clostridium ghonii Spore on Colon Cancer Tumor-Bearing Mouse Model

Materials: Clostridium ghonii spore dried powder for injection: an MW-DCG-LCv-26 strain was deposited in the National Measurement Institute, Australia, with a date of deposit of Apr. 18, 2012 and a deposit number of V12/001486 and developed by Shandong Xinchuang Biotechnology Co., Ltd. The Clostridium ghonii spore freeze-dried powder for injection used a Clostridium ghonii spore as an active ingredient and 1% sucrose as an auxiliary material. A freeze-drying procedure included: freezing at −40° C. for 4 h, vacuumizing at −35° C. for 10 min, and freeze-drying at −30° C. for 10 min, −25° C. for 10 min, −20° C. for 26 h, −15° C. for 2 h, −10° C. for 10 min, −5° C. for 10 min, 0° C. for 10 min, 10° C. for 2 h, 15° C. for 10 min, 20° C. for 3 h and 27° C. for 3 h. The freeze-dried powder had a specification of 1×10⁸ CFU/bottle. A reference substance freeze-dried powder had a batch number: 201803001F, and was developed by Shandong Xinchuang Biotechnology Co., Ltd., by the above freeze-drying procedure with 1 mL of a 1% sucrose solution. A 0.9% sodium chloride injection had a batch number: 1803122161, and was commercially available in CISEN Pharmaceutical Co., LTD. Sterile water for injection had a batch number: 1704242163, and was commercially available in CISEN Pharmaceutical Co., LTD. CT26.WT colon cancer cells had a number of 3131C0001000800037 and were deposited in Cell Resource Center, Shanghai Institutes for Biological Sciences, Chinese Academy of Sciences. BALB/c female mice, with animal qualification certificate numbers: No. 11401300088557 (120 mice), No. 11401300089721 (60 mice), and No. 11401300089721 (60 mice), were commercially available in Beijing HFK Bioscience Co., Ltd.

Methods: the CT26.WT cells were recovered and passaged to the required number, and prepared into a cell suspension for inoculation with a concentration of 7.5×10⁶ cells/mL and a cell viability over 90% to establish a subcutaneous xenograft model for colon cancer in the BALB/c mice. 0.2 mL of the cell suspension was subcutaneously inoculated on right forelimbs of the mice. Experimental animals with a tumor volume of about 0.30 cm³ were selected for an experiment in about 10 days. The tumor-formation conforming mice were randomly divided into 4 groups by lottery: control group (Control), low-dose treatment group (L C. ghonii), medium-dose treatment group (M C. ghonii), and high-dose treatment group (H C. ghonii), with 8 mice in each group. The Clostridium ghonii spore dried powder was first re-dissolved with 0.1 mL of sterile water for injection and then prepared into suspensions with concentrations of 5×10⁷ cfu/mL, 1×10⁸ cfu/mL, and 2×10⁸ cfu/mL separately using a sodium chloride injection with a mass percentage of 0.9%. The mice were intratumorally injected with 0.1 mL of the suspensions separately once every other day for a total of 5 times at a dose of 5×10⁶ cfu/time, 1×10⁷ cfu/time, and 2×10⁷ cfu/time separately. The mice in the control group were injected with a reference substance at a concentration the same as that of the suspension in the high-dose treatment group. After the administration began, animal behaviors, death, near-death, and other clinical symptoms were observed every day. All surviving animals were dissected on the 2nd day after the last administration. Tumor weight was weighed and a tumor inhibition rate based on the tumor weight was calculated. Tumor inhibition rate based on tumor weight (IR_{TW} %)=(average tumor weight of control group-average tumor weight of experimental group)/average tumor weight of control group×100%. 3 batches of independent repeated experiments were conducted separately using the experimental method.

Results: during the experiment, all animals in 3 batches of the experiments did not show near-death/death. At the end of the experiments, the average tumor weight of the tumor-bearing mice in each group and the tumor inhibition rate in the treatment groups in 3 batches of the experiments were shown in Table 1 and Table 2 separately.

TABLE 1
Average tumor weight of tumor-bearing mice in each group (g)

| Experiment | Control | L C. ghonii | M C. ghonii | H C. ghonii |
|---|---|---|---|---|
| batch | group | group | group | group |
| 1 | 2.85 ± 0.59 | 1.98 ± 0.76 | 1.84 ± 0.67 | 2.45 ± 0.34 |
| 2 | 4.15 ± 0.51 | 2.00 ± 0.61 | 2.23 ± 0.78 | 2.50 ± 0.71 |
| 3 | 2.69 ± 0.52 | 1.73 ± 0.56 | 1.72 ± 0.42 | 1.93 ± 0.39 |

TABLE 2
Tumor inhibition rate based on tumor weight (%) in treatment groups

| Experiment | L C. ghonii | M C. ghonii | H C. ghonii |
|---|---|---|---|
| batch | group | group | group |
| 1 | 30.40 | 35.58 | 14.12 |
| 2 | 51.90 | 46.17 | 39.79 |
| 3 | 35.46 | 35.97 | 28.06 |

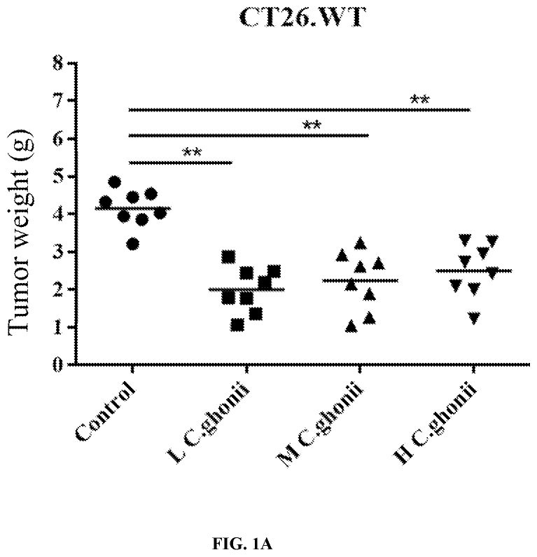

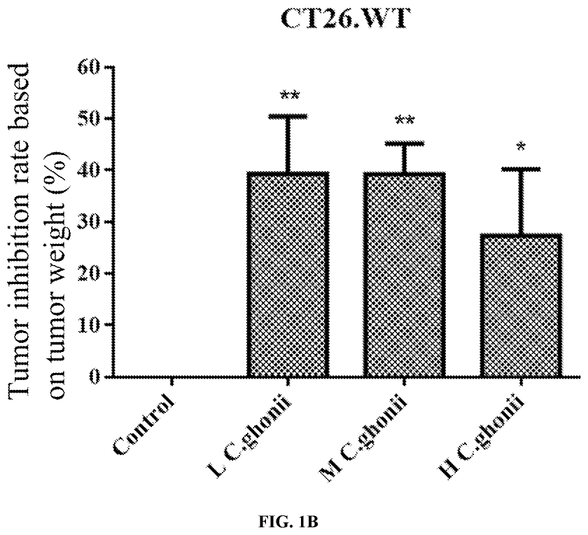

The tumor weight of the mice in the treatment groups in each batch was separately smaller than that of mice in the Control group, and a best anti-tumor effect was shown in the M C. ghonii group (FIG. 1A). An inhibitory effect on tumor growth was shown in the treatment groups of each dose, a best tumor inhibition rate based on tumor weight was shown in the M C. ghonii group in experiment batches 1 and 3, and a best tumor inhibition rate based on tumor weight was shown in the L C. ghonii group in experimental batch 2. Statistical analysis based on the results of 3 batches of the experiments showed that the tumor inhibition rate based on tumor weight in the H C. ghonii group was significantly different from that in the Control group (P<0.05) and the tumor inhibition rate based on tumor weight in the L C. ghonii group and the M C. ghonii group was separately extremely significant different from that in the Control group (both P<0.01) as shown in FIG. 1B.

In conclusion, the Clostridium ghonii spore freeze-dried powder had an inhibitory effect on growth of colon cancer and the effect was better in the M C. ghonii group (1×10⁷ cfu/time) than in other treatment groups. Therefore, a preferred dose of the C. ghonii combined with pembrolizumab in a next experiment was 1×10⁷ cfu/time.

### Example 2 Therapeutic Effect of Clostridium ghonii Spore Combined with Pembrolizumab on PD-1 Humanized Transgenic Colon Cancer Tumor-Bearing Mouse Model

Materials: Clostridium ghonii spore dried powder for injection was developed by Shandong Xinchuang Biotechnology Co., Ltd., and a preparation method was the same as that in the Materials of Example 1; a pembrolizumab injection had a batch number of S001188 and a specification of 100 mg/4 mL, and was commercially available in Merck & Co., Inc.; reference substance freeze-dried powder had a batch number of 201910002F and 201803001F and was developed by Shandong Xinchuang Biotechnology Co., Ltd., and a preparation method was the same as that in the Materials of Example 1; a 0.9% sodium chloride injection had a batch number of 1809282161 and was commercially available in CISEN Pharmaceutical Co., LTD.; sterile water for injection had a batch number of 1902212162 and was commercially available in CISEN Pharmaceutical Co., LTD.; MC38 colon cancer cells had a Art. No. T1917 and were commercially available in Abm Biotechnology Co., Ltd.; and C57BL/6 PD-1 humanized genetically engineered mice, with an animal certificate number of No. 20170010002500 (90 mice), No. 312024300008757 (35 mice) and No. 20170010001319 (50 mice), were commercially available in Shanghai Model Organisms.

Methods: the MC38 cells were recovered and passaged to the required number, and prepared into a cell suspension for inoculation with a concentration of 7.5×10⁶ cells/mL and a cell viability over 90% to establish a subcutaneous xenograft model for colon cancer in the C57BL/6 PD-1 humanized genetically engineered mice. 0.2 mL of the cell suspension was subcutaneously inoculated on right forelimbs of the mice. Experimental animals with a tumor volume larger than 0.15 cm³ were selected for an experiment in about 10 d. The screened conforming mice were randomly divided into 4 groups by lottery: control group (Control), Clostridium ghonii spore treatment group (C. ghonii), pembrolizumab group, and Clostridium ghonii spore combined pembrolizumab group (C. Ghonii+Pembrolizumab), with more than or equal to 5 mice in each group. The Clostridium ghonii spore dried powder was first re-dissolved with 0.1 mL of sterile water for injection and then prepared into a suspension with a concentration 1×10⁸ cfu/mL using a sodium chloride injection with a mass percent of 0.9%. The mice were intratumorally injected with 0.1 mL of the suspension at a dose of 1×10⁷ cfu/time in the C. ghonii group and the C. Ghonii+Pembrolizumab group and at a dose of 0 cfu/time in the Control group and the Pembrolizumab group once every other day for a total of 6 times. The pembrolizumab injection was diluted into a 1 mg/mL working solution using the 0.9% sodium chloride injection. The mice were intraperitoneally injected with 0.2 mL of the working solution in the Pembrolizumab group and the C. Ghonii+Pembrolizumab group at 0.2 mg/time and intraperitoneally injected with 0.2 mL of the 0.9% sodium chloride injection twice every week for a total of 4 times.

The mice were firstly administrated with the Clostridium ghonii spore and then a PD-1 antibody in the following administration sequence: Clostridium ghonii spore was injected every other day from the 1st day; and pembrolizumab was injected on the 3rd, 6th, 9th and 13rd day separately.

Observation and evaluation indicators: after the administration began, animal behaviors, death or near-death were observed every day. A tumor volume was observed every other 1-2 d and a tumor inhibition rate was calculated based on the tumor volume measured at a last time of the experiment. All surviving animals were dissected on the 2nd day after the last administration. Tumor weight was weighed and a tumor inhibition rate based on the tumor weight was calculated.

Tumor inhibition rate based on tumor weight(IR_{TW}%)=(average tumor weight of control group-average tumor weight of experimental group)/average tumor weight of control group×100%.

Tumor inhibition rate based on tumor volume(IR_{TW}%)=1−(average tumor volume of experimental group at a beginning-average tumor volume of experimental group at an end)/(average tumor volume of control group at a beginning-average tumor volume of control group at an end)×100%.

Cure rate (%)=number of cured animals in each group/total number of experimental animals in each group×100%(at an end of the experiment).

3 batches of independent repeated experiments were conducted separately using the experimental method.

Results: during the experiment, 1 mouse in the control group of experiment batch 1 was dead, but mice in other batches or other groups did not show death or near-death.

1. Average Tumor Weight and Tumor Inhibition Rate of Tumor-Bearing Mice in Each Group

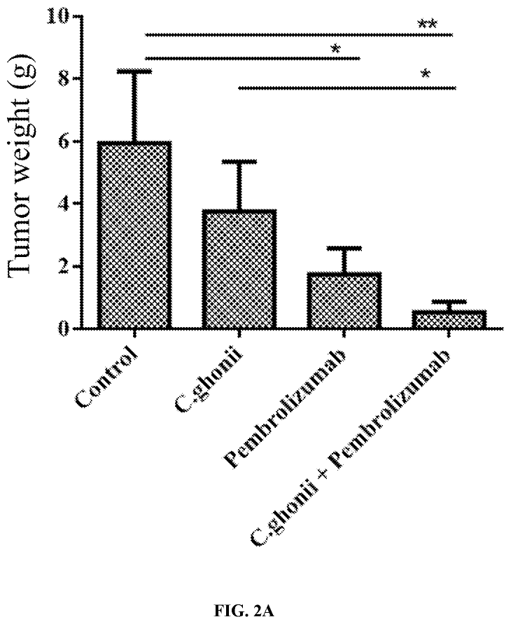

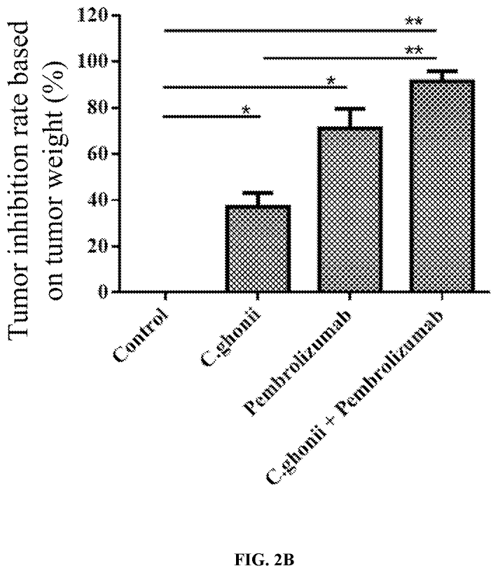

The average tumor weight of the tumor-bearing mice in each group in 3 batches of the experiments was shown in Table 3 separately. The average tumor weight of the mice in each treatment group was smaller than that of the mice in the Control group, and the tumor weight of the mice in the C. ghonii+Pembrolizumab group was smaller than that of the mice in any single drug treatment group, and was extremely significant lower than that of mice in the Control group (P<0.01). The average tumor weight of the mice in the C. ghonii+Pembrolizumab group was also significantly smaller than that of mice in the single C. ghonii group and the single Pembrolizumab group separately (both P<0.05) (FIG. 2A).

TABLE 3
Average tumor weight of tumor-bearing mice in each group (g)

| | | | | C. ghonii + |
|---|---|---|---|---|
| Experiment | Control | C. ghonii | Pembrolizumab | Pembrolizumab |
| batch | group | group | group | group |
| 1 | 3.46 ± 1.33 | 2.24 ± 0.74 | 0.83 ± 0.74 | 0.18 ± 0.19 |
| 2 | 7.97 ± 2.46 | 5.42 ± 1.70 | 1.92 ± 1.42 | 0.56 ± 0.45 |
| 3 | 6.41 ± 1.46 | 3.60 ± 1.09 | 2.48 ± 1.40 | 0.86 ± 0.79 |

Statistical analysis based on the results of 3 batches of the experiments showed that an inhibitory effect on tumor growth was exhibited in each treatment group with a tumor inhibition rate above 30%. The tumor inhibition rate: C. ghonii+Pembrolizumab group>Pembrolizumab group>C. ghonii group (FIG. 2B). After administration of the pembrolizumab at a same dose, the tumor inhibition rate in the C. ghonii+Pembrolizumab group increased by approximately 20%, 20%, and 25% separately compared with that in the single Pembrolizumab group (Table 4).

TABLE 4
Tumor inhibition rate based on tumor weight (%) in treatment groups

| Experiment | C. ghonii | Pembrolizumab | C. ghonii + |
|---|---|---|---|
| batch | group | group | Pembrolizumab group |
| 1 | 35.33 | 76.04 | 94.76 |
| 2 | 32.03 | 75.95 | 92.95 |
| 3 | 43.82 | 61.27 | 86.61 |

2. Average Tumor Volume and Tumor Inhibition Rate of Tumor-Bearing Mice in Each Group

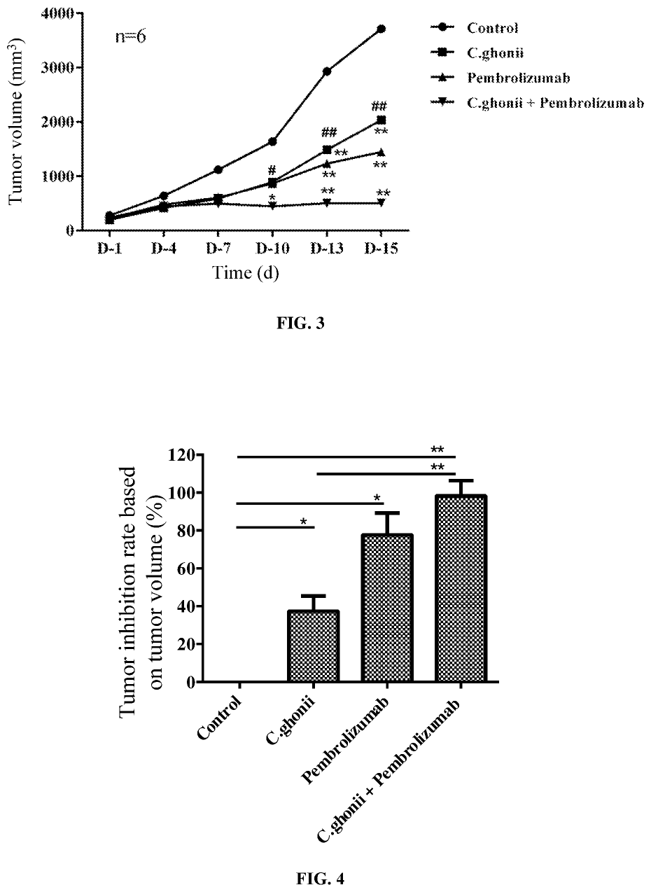

Before the experiment began, there was no significant difference in the average tumor volume of mice in each group of each batch (all P>0.05). At an end of administration of the tumor-bearing mice in experiment batch 3, the tumor volume of mice in the C. ghonii group, the Pembrolizumab group, and the C. ghonii+Pembrolizumab group was significantly smaller than that of mice in the Control group separately (P=0.001, P=0.000053, and P=0.000). The tumor volume of the mice in the C. ghonii+Pembrolizumab group was obviously smaller than that of mice in the C. ghonii group and the Pembrolizumab group. Besides, on the 10th day, there was a significant difference in the average tumor volume between the C. ghonii+Pembrolizumab group and the Control group. Meanwhile, the tumor volume of the mice in the C. ghonii+Pembrolizumab group was nearly 1 time smaller than that of the mice in the Pembrolizumab group (FIG. 3).

The tumor inhibition rate was calculated based on the tumor volume. During 3 batches of the experiments, the tumor inhibition rate in the C. ghonii+Pembrolizumab group was higher than that in the other groups (FIG. 4).

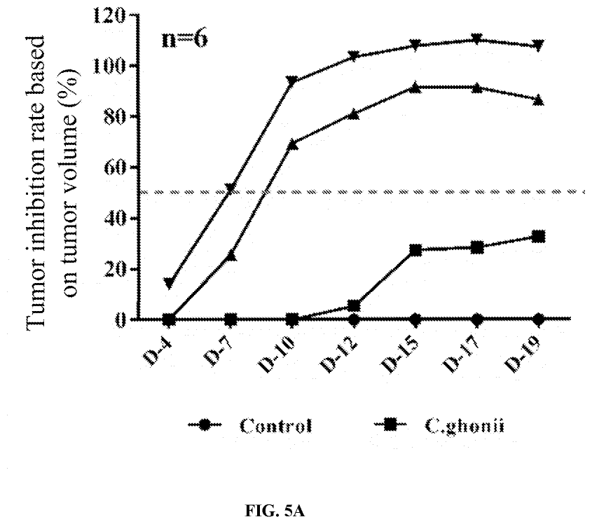

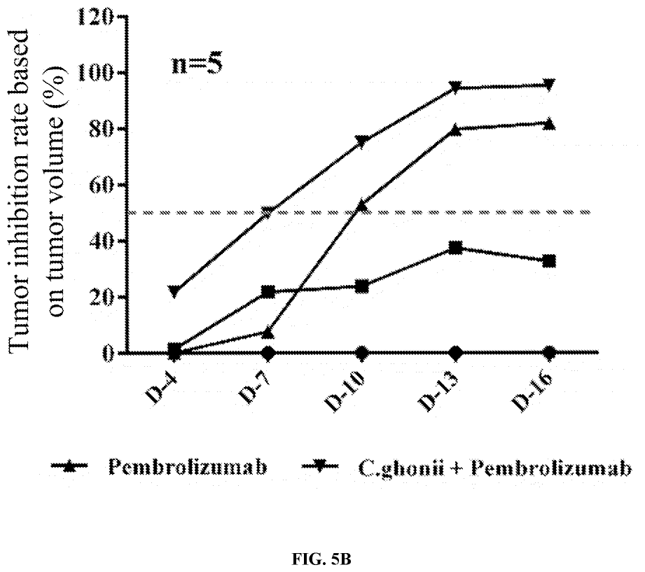

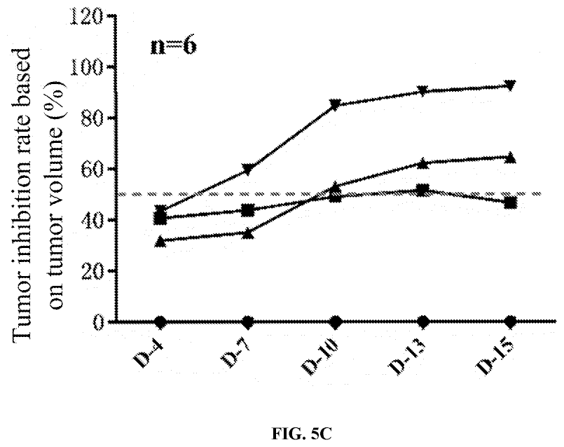

On the 7th day after administration in 3 batches of the experiments, the pembrolizumab was administered twice. After the pembrolizumab was administered at a dose of 0.4 mg, the tumor inhibition rate in the C. ghonii+Pembrolizumab group was 51.29% (greater than 50%), 49.78% (approximately 50%), and 59.33% (greater than 50%) separately, while the tumor inhibition rate in the single Pembrolizumab group was 25.67%, 7.68%, and 35.10% separately. After the mice in the single Pembrolizumab alone group were administered 3 times with pembrolizumab at a total dose of 0.6 mg, the tumor inhibition rate reached that of the C. ghonii+Pembrolizumab group when the pembrolizumab was administered twice (at a total dose of 0.4 mg), and were 69.31%, 52.83%, and 53.08% separately (FIGS. 5A-5C). It can be seen that an anti-tumor effect of the pembrolizumab was significantly improved in the C. ghonii+Pembrolizumab group. The dose of the pembrolizumab required to obtain a same therapeutic effect in the C. ghonii+Pembrolizumab group was reduced by 50%, thus high efficiency and low toxicity were reached.

3. Cure Rate

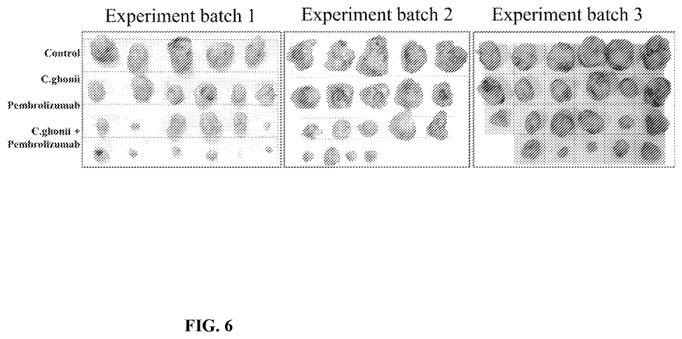

About 20% (⅙, ⅕, and ⅙) of mouse tumors were completely eliminated in 3 batches of the experiments in the C. ghonii+Pembrolizumab group (Table 5), and no tumor growth was seen at the end of the experiments. But no complete tumor elimination was shown in the Pembrolizumab group and the C. ghonii group, that is, the cure rate was 0%. This may due to the fact that a Clostridium ghonii spore germinated in a hypoxic area of a tumor, could affect immunogenicity of a tumor microenvironment (TME) by various ways, converted an immunosuppressive state of the TME into an immune-activated state, adjusted the immunosuppressive TME, and broke an immune tolerance. Combined with the pembrolizumab, a therapeutic effect of the PD-1 antibody can be further enhanced, and about 20% of the mice were cured. The Clostridium ghonii was expected to become an excellent “sensitizer” for immunotherapy of a patient with a tumor. Morphology of tumors of each group was shown in FIG. 6.

TABLE 5
Cure rate (%)

| | | | | C. ghonii + |
|---|---|---|---|---|
| Experiment | Control | C. ghonii | Pembrolizumab | Pembrolizumab |
| batch | group | group | group | group |
| 1 | 0 | 0 | 0 | 16.7 |
| 2 | 0 | 0 | 0 | 20.0 |
| 3 | 0 | 0 | 0 | 16.7 |

### Example 3 Influence of Administration Sequence of Clostridium ghonii Spore and Pembrolizumab on Therapeutic Effect on PD-1 Humanized Transgenic Colon Cancer Tumor-Bearing Mouse Model

Materials: Clostridium ghonii spore dried powder for injection was developed by Shandong Xinchuang Biotechnology Co., Ltd., and a preparation method was the same as that in the Materials of Example 1; a pembrolizumab injection had a batch number of S006648 and a specification of 100 mg/4 mL, and was commercially available in Merck & Co., Inc.; reference substance freeze-dried powder had a batch number of 201910002F and was developed by Shandong Xinchuang Biotechnology Co., Ltd., and a preparation method was the same as that in the Materials of Example 1; a 0.9% sodium chloride injection had a batch number of J18070104 and was commercially available in Shandong Hualu Pharmaceutical Co. Ltd.; sterile water for injection had a batch number of 1902212162 and was commercially available in CISEN Pharmaceutical Co., LTD.; MC38 colon cancer cells had aArt. No. T1917 and were commercially available in Abm Biotechnology Co., Ltd.; and C57BL/6 PD-1 humanized genetically engineered mice, with an animal certificate number of No. 20170010002500 (90 mice), were commercially available in Shanghai Model Organisms.

Methods: A subcutaneous xenograft model for MC38 colon cancer in the C57BL/6 PD-1 humanized genetically engineered mice was established with a same method as Example 2. The successfully modeled mice were randomly divided by lottery into A: control group (Control), B: Clostridium ghonii spore group (C. ghonii), C: Pembrolizumab group, D: first Pembrolizumab and then C. ghonii group, E: first C. ghonii and then Pembrolizumab group, F: C. ghonii+Pembrolizumab simultaneous group, 8 mice in each group; the C. ghonii was administrated at a dose of 0 cfu/time, 1×10⁷ cfu/time, 0 cfu/time, 1×10⁷ cfu/time, 1×10⁷ cfu/time and 1×10⁷ cfu/time correspondingly, with an administration volume of 0.1 mL/time, once every other day; and 0.2 mL of 1 mg/mL of a Pembrolizumab injection was administered intraperitoneally, at a dose of 0.2 mg/time, once every other day. An administration process in a first stage was shown in Table 6.

TABLE 6
Administration process in first stage

| | First administration | | Second administration | | Third administration | | Last administration | |
|---|---|---|---|---|---|---|---|---|
| | C. | | C. | | C. | | C. | |
| Grouping | ghonii | Pembrolizumab | ghonii | Pembrolizumab | ghonii | Pembrolizumab | ghonii | Pembrolizumab |
| A | | | | | | | | |
| B | √ | | √ | | √ | | √ | |
| C | | √ | | √ | | √ | | √ |
| D | | √ | √ | √ | √ | √ | | √ |
| E | √ | | √ | √ | √ | √ | √ | |
| F | √ | √ | √ | √ | √ | √ | √ | √ |

After the administration in the first stage, the mice were observed for 6 days. The administration process was repeated in a second stage as in the first stage.

Observation and evaluation indicators: after the administration began, animal behaviors, death or near-death were observed every day. A tumor volume was observed every other 1-2 d and a tumor inhibition rate was calculated based on the tumor volume measured at a last time of the experiment. All surviving animals were dissected on the 8th day after the last administration of the second stage. Tumor weight was weighed and a tumor inhibition rate based on the tumor weight was calculated.

Tumor inhibition rate based on tumor weight(IR_{TW}%)=(average tumor weight of control group-average tumor weight of experimental group)/average tumor weight of control group×100%.

Cure rate (%)=number of cured animals in each group/total number of experimental animals in each group×100%(at an end of the experiment).

Results: during the experiment, 4 mouse in the Control group were dead due to oversized or ruptured tumors, but mice in other groups did not show death or near-death.

1. Tumor Weight and Tumor Inhibition Rate

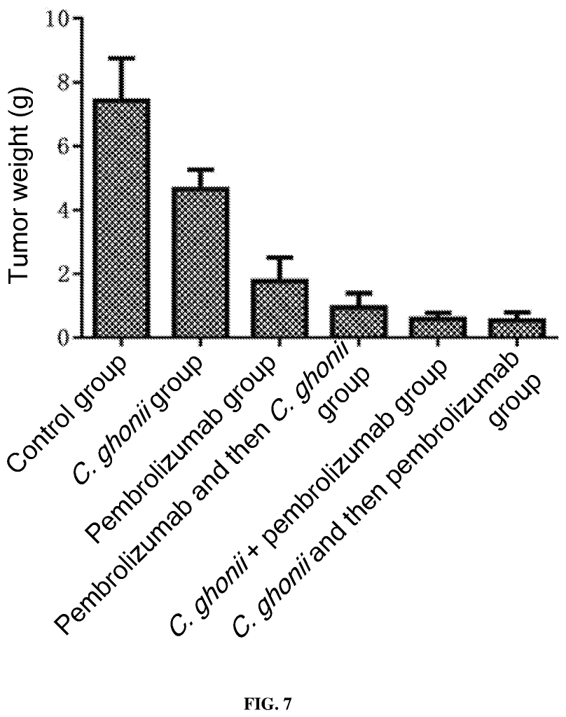

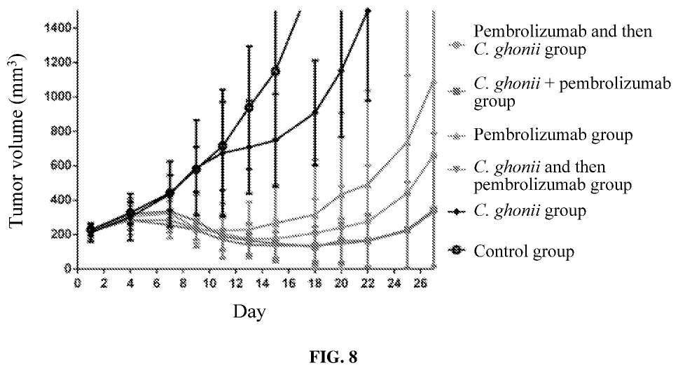

Compared with the Control group, the tumor weight in the C. ghonii group, the Pembrolizumab group, the first Pembrolizumab and then C. ghonii group, the first C. ghonii and then Pembrolizumab group, and the C. ghonii+Pembrolizumab simultaneous group was separately significantly smaller than that in the Control group (P=0.005, P=0.000, P=0.000, P=0.000, and P=0.000). The tumor weight of mice in each group was shown Table 7 and FIG. 7.

TABLE 7
Tumor weight

| | | | | First | First C. ghonii | |
|---|---|---|---|---|---|---|
| | | | | Pembrolizumab | and then | C. ghonii + |
| | Control | C. ghonii | Pembrolizumab | and then C. | Pembrolizumab | Pembrolizumab |
| Grouping | group | group | group | ghonii group | group | group |
| Tumor | 7.43 ± 2.66 | 4.65 ± 1.72 | 1.77 ± 2.08** | 0.95 ± 1.29** | 0.54 ± 0.69** | 0.58 ± 0.56** |
| weight | | | | | | |
| (g) | | | | | | |

Note:
**indicates P < 0.01 compared with the Control group.

The tumor inhibition rate was calculated according to tumor weight. The results showed that the tumor inhibition rate in the C. ghonii group, the Pembrolizumab group, the first Pembrolizumab and then C. ghonii group, the first C. ghonii and then Pembrolizumab group, and the C. ghonii+Pembrolizumab group was 37.39%, 76.20%, 87.26%, 92.71%, and 92.19% separately. A significant inhibitory effect on tumor growth was shown in each treatment group (Table 8).

TABLE 8
Tumor inhibition rate

| | | | First | First C. ghonii | |
|---|---|---|---|---|---|
| | | | Pembrolizumab | and then | C. ghonii + |
| | C. ghonii | Pembrolizumab | and then | Pembrolizumab | Pembrolizumab |
| Grouping | group | group | C. ghonii group | group | group |
| Tumor | 37.39 | 76.20 | 87.26 | 92.71 | 92.19 |
| inhibition | | | | | |
| rate (%) | | | | | |

2. Tumor Volume and Tumor Inhibition Rate

Before the treatment, there was no significant difference in the tumor volume of the tumor-bearing mice in each group (all P>0.05). At the end of the administration, the tumor volume of the mice in the C. ghonii group, the Pembrolizumab group, the first Pembrolizumab and then C. ghonii group, the first C. ghonii and then Pembrolizumab group, and C. ghonii+Pembrolizumab simultaneous group was separately significantly smaller than that of the mice in the Control group (P=0.002, P=0.000, P=0.000, P=0.000, and P=0.000, FIG. 8). The tumor inhibition rate: first C. ghonii and then Pembrolizumab group>C. ghonii+Pembrolizumab simultaneous group>first Pembrolizumab and then C. ghonii group>Pembrolizumab group>Control group as shown in Table 9.

TABLE 9
Tumor inhibition rate

| | | | | First | First C. ghonii | |
|---|---|---|---|---|---|---|
| | | | | Pembrolizumab | and then | C. ghonii + |
| | Control | C. ghonii | Pembrolizumab | and then C. | Pembrolizumab | Pembrolizumab |
| Grouping | group | group | group | ghonii group | group | group |
| Tumor | — | 40.99 | 79.07 | 87.36 | 93.62 | 93.42 |
| inhibition | | | | | | |
| rate (%) | | | | | | |

3. Cure Rate

During the experiment, the cure rate of the mice in each group was shown in Table 10.

TABLE 10
Cure rate

| | | | | First | First C. ghonii | |
|---|---|---|---|---|---|---|
| | | | | Pembrolizumab | and then | C. ghonii + |
| | Control | C. ghonii | Pembrolizumab | and then C. | Pembrolizumab | Pembrolizumab |
| Grouping | group | group | group | ghonii group | group | group |
| cure rate | 0 | 0 | 0 | 12.5 | 25 | 12.5 |
| (%) | | | | | | |

The tumor cure rate of the mice in the first C. ghonii and then Pembrolizumab group was significantly higher than that of the mice in other groups, and was twice the cure rate of the first Pembrolizumab and then C. ghonii group and C. ghonii+Pembrolizumab simultaneous group. This may be due to the fact that intratumoral administration of the Clostridium ghonii spore was effective in lysing tumor tissue without distinguishment and destroying the TME. At the same time, oncolysis by the bacteria could recruit immune cells to infiltrate into the TME, and changed the infiltration degree and the composition of the immune cells in the TME. A cell lysate would attract a large number of CD8⁺ T cells to aggregate. The PD-1 antibody treatment was conducted after the oncolysis by the bacteria, avoids immunosuppression in immunotherapy, launched a large-scale attack of T cells on tumor cells, and thus plays a “double” anti-cancer effect. Therefore, the administration sequence was also critical to the therapeutic effect on the tumors when the oncolysis by the bacteria combined with the immunosuppressive agent.

In conclusion, the first Clostridium ghonii spore treatment then combined with the immunotherapy could enhance an anti-tumor effect, reduced a dose of the immune drug, and thus was efficient and low-toxic.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1-3. (canceled)

4. A drug for treating colon cancer, comprising active ingredients of a Clostridium ghonii spore and pembrolizumab.

5. The drug according to claim 4, wherein the Clostridium ghonii is a Clostridium ghonii MW-DCG-LCv-26 strain or a strain obtained after domestication of the Clostridium ghonii; and the Clostridium ghonii MW-DCG-LCv-26 strain is deposited in the National Measurement Institute, Australia, with a date of deposit of Apr. 18, 2012 and a deposit number of V12/001486.

6. The drug according to claim 5, wherein the strain obtained after domestication of the Clostridium ghonii is an MW-DCG-HNCv-18 strain deposited in the National Measurement Institute, Australia, with a date of deposit of Apr. 18, 2012 and a deposit number of V12/001485.

7. The drug according to claim 5, wherein the strain obtained after domestication of the Clostridium ghonii is an MW-DCG-CCv-17 strain deposited in the National Measurement Institute, Australia, with a date of deposit of Apr. 18, 2012 and a deposit number of V12/001487.

8. The drug according to claim 4, wherein in the drug, the Clostridium ghonii spore is a Clostridium ghonii spore freeze-dried powder for injection with an auxiliary material of 1% sucrose.

9. The drug according to claim 8, wherein a freeze-drying procedure of the Clostridium ghonii spore freeze-dried powder for injection comprises: maintaining −40° C. for 4 h, vacuumizing, and freeze-drying at −35° C. for 10 min, −30° C. for 10 min, −25° C. for 10 min, −20° C. for 26 h, −15° C. for 2 h, −10° C. for 10 min, −5° C. for 10 min, 0° C. for 10 min, 10° C. for 2 h, 15° C. for 10 min, 20° C. for 3 h and 27° C. for 3 h.

10. The drug according to claim 4, wherein the pembrolizumab is a PD-1 antibody injection.

11. The drug according to claim 4, wherein every 1×10⁷ CFU of the Clostridium ghonii spore is combined with 0.2 mg of a pembrolizumab solution at a concentration of 1 mg/mL.

12. The drug according to claim 11, wherein the pembrolizumab solution has a solvent of a sodium chloride injection with a mass percentage of 0.9%.

13. The drug according to claim 11, wherein the Clostridium ghonii spore is the Clostridium ghonii spore freeze-dried powder for injection, and the Clostridium ghonii spore freeze-dried powder for injection has a solvent of sterile water for injection and a sodium chloride injection with a mass percentage of 0.9%.

14. A method for treating colon cancer by using the drug according to claim 4.

15. The method according to claim 14, wherein the Clostridium ghonii spore is administered before the pembrolizumab.

16. The method according to claim 14, wherein the Clostridium ghonii is a Clostridium ghonii MW-DCG-LCv-26 strain or a strain obtained after domestication of the Clostridium ghonii; and the Clostridium ghonii MW-DCG-LCv-26 strain is deposited in the National Measurement Institute, Australia, with a date of deposit of Apr. 18, 2012 and a deposit number of V12/001486.

17. The method according to claim 16, wherein the strain obtained after domestication of the Clostridium ghonii is an MW-DCG-HNCv-18 strain deposited in the National Measurement Institute, Australia, with a date of deposit of Apr. 18, 2012 and a deposit number of V12/001485.

18. The method according to claim 16, wherein the strain obtained after domestication of the Clostridium ghonii is an MW-DCG-CCv-17 strain deposited in the National Measurement Institute, Australia, with a date of deposit of Apr. 18, 2012 and a deposit number of V12/001487.

19. The method according to claim 14, wherein in the drug, the Clostridium ghonii spore is a Clostridium ghonii spore freeze-dried powder for injection with an auxiliary material of 1% sucrose.

20. The method according to claim 19, wherein a freeze-drying procedure of the Clostridium ghonii spore freeze-dried powder for injection comprises: maintaining −40° C. for 4 h, vacuumizing, and freeze-drying at −35° C. for 10 min, −30° C. for 10 min, −25° C. for 10 min, −20° C. for 26 h, −15° C. for 2 h, −10° C. for 10 min, −5° C. for 10 min, 0° C. for 10 min, 10° C. for 2 h, 15° C. for 10 min, 20° C. for 3 h and 27° C. for 3 h.

21. The method according to claim 14, wherein the pembrolizumab is a PD-1 antibody injection.

22. The method according to claim 14, wherein every 1×10⁷ CFU of the Clostridium ghonii spore is combined with 0.2 mg of a pembrolizumab solution at a concentration of 1 mg/mL.

23. The method according to claim 22, wherein the pembrolizumab solution has a solvent of a sodium chloride injection with a mass percentage of 0.9%.
